(19) United States
(12) Patent Application Publication
(10) Pub. No.: US 2008/0086111 A1
(43) Pub. Date: Apr. 10, 2008

(54) FLUID DELIVERY SYSTEMS AND VOLUME METERING IN CELL DELIVERY

(75) Inventors: Kevin P. Cowan, Allison Park, PA (US); Frederick W. Trombley, Gibsonia, PA (US)

Correspondence Address: GREGORY L BRADLEY;MEDRAD INC, ONE MEDRAD DRIVE, INDIANOLA, PA, 15051, US

(73) Assignee: MEDRAD, INC., Indianola, PA (US)

(21) Appl. No.: 11/869,268

(22) Filed: Oct. 9, 2007

Related U.S. Application Data
(60) Provisional application No. 60/828,713, filed on Oct. 9, 2006.

(51) Int. Cl. A61M 5/145

(52) U.S. Cl. 604/522; 604/153

(57) ABSTRACT

A fluid delivery system includes a source of an injection fluid including a source outlet. The system also includes a control system including a control system inlet in fluid connection with the source outlet, a control system outlet and, alternatively, an actuator. The control system is adapted to deliver a predetermined amount of fluid via the control system outlet upon activation of the actuator or modification of the control system. In several embodiments, the injection fluid in the source is pressurized. The source can, for example, include a plunger slidably disposed therein and a force application mechanism to place force upon the plunger and pressurize the fluid within the source. The control system can further include a metering volume in fluid connection with a valve system. The metering volume can include a plunger slidably disposed therein.

### CROSS-REFERENCE TO RELATED APPLICATION

This application is related to U.S. patent application Ser. No. 11/460,635, filed Jul. 28, 2006, U.S. Provisional Patent Application Ser. No. 60/771,206, filed Feb. 7, 2006, U.S. Provisional Patent Application Ser. No. 60/742,224, filed Dec. 5, 2005, and U.S. Provisional Patent Application Ser. No. 60/734,035, filed Nov. 4, 2005, the disclosures of which are incorporated herein by reference.

### BACKGROUND

The present disclosure relates to fluid delivery systems and, particularly to fluid delivery systems for the delivery of agents such as therapeutic agents to tissue and, even more particularly, to the fluid delivery systems suitable to for repeated delivery of a predetermined volume of fluid to tissue (for example, in cell therapy).

The following information is provided to assist the reader to understand the disclosure described below and the environment in which it will typically be used. The terms used herein are not intended to be limited to any particular narrow interpretation unless clearly stated otherwise in this document. References set forth herein may facilitate understanding of the present disclosure or the background of the present disclosure. The disclosure of all references cited herein are incorporated by reference.

The treatment of disease by the injection of living cells into a body is expanding rapidly. There are many types of cells being used to treat an equally diverse set of diseases, and both types of cells and disease conditions are expanding rapidly. Xenogeneic cell therapies involve implantation of cells from one species into another. Allogeneic cell therapies involve implantation from one individual of a species into another individual of the same species. Autologous cell therapies involve implantation of cells from one individual into the same individual.

In an example of an allogeneic cell therapy, current phase II clinical trials of SPHERAMINE® by Titan Pharmaceutical of San Francisco, Calif. and Schering AG of Berlin, Germany, retinal pigment epithelial cells are harvested from eyes in eye banks, multiplied many fold in culture medium and placed on 100 micrometer diameter gelatin spheres. The spherical microscopic carriers or microcarriers greatly enhance the cells' survival when injected in the brain. The carriers are injected through needles into the putamen in the brain. The animal precursor work is described in several patents, including U.S. Pat. Nos. 6,060,048, 5,750,103, and 5,618,531, the disclosures of which are incorporated herein by reference. These patents describe many types of cells, carriers, and diseases that can be treated via the disclosed methods. In a rat, about 20 microliters (ul) of injected cells on carriers is sufficient to restore dopamine production to a damaged rat brain. The therapy was injected at the rate of 4 ul/min. This dosage scales to a total injected volume of 0.5 ml in the human brain, although it will have to be distributed over a larger region, probably via multiple individual injections on the order of the 20 ul mentioned above. Cell therapies for the brain and nervous system are discussed further below.

An example of an autologous cell therapy involves the harvesting of mesenchymal stem cells from a patient's bone marrow, concentration of the stem cells, and injection of the cells and other blood components into the heart muscle during open-heart surgery. Further examples include catheter delivered cell therapies, especially to the heart, laparoscopic delivered therapies, and transcutaneous therapies.

In external cell therapy for the heart, volumes of about 0.5 to 1.0 ml are injected into a beating heart. A multi-milliliter syringe is used to hold and deliver the injectate under manual activation. A challenge is presented in that when the heart is contracting, during systole, the tissue becomes relatively hard and tense. In diastole, the tissue relaxes. It is very difficult for a human to time and control a hand injection so that the proper volume is injected all in one period of diastole. In practice, an indeterminate amount of the injectate can squirt or leak out the needle track and is presumably wasted. In addition, it is desirable to uniformly and thoroughly treat the target areas of the heart, and to avoid puncturing the major blood vessels traversing the outside of the heart. These results can also be difficult to achieve with current manual injection practices. With the current state of practice, scar tissue is not injected or treated because it does not respond well, and the growth that does occur can sometimes create dangerous electrical conduction abnormalities.

Cell therapies are generally delivered by hand injection through a needle or catheter. The benefits of hand or manual injection are conceptual simplicity and familiarity for the doctor. However the simplicity is misleading. Many of the parameters of the injection are not and cannot be controlled or even repeated by that doctor, let alone by other doctors. Flow rate is, for example, very difficult to control manually, especially at low flow rates. The stick slip friction of normal syringes exacerbates this problem. Volume accuracy depends upon manual reading of gradations, which is physically difficult while squeezing the syringe and susceptible to human perceptual or mathematical errors. The use of common infusion pumps limits delivery to generally slow and very simple fluid deliveries. Infusion pumps, though, have no ability to provide automatic response or action to the injection based upon any physiological or other measurement or feedback.

Tremendous variations in manually controlled injectate delivery can produce proportionally wide variations in patient outcomes. In clinical trials, this variation is undesirable because it increases the number of patients and thus also increases the cost and time needed to establish efficacy. In long term therapeutic use, such variation remains undesirable as some people can receive suboptimal treatment.

FIG. 1 illustrates the current manual state of the art. Cells are taken from a bag or other storage or intermediate container and loaded into a syringe. This procedure involves making and breaking fluid connections in the room air which can compromise sterility, or requires a special biological enclosure to provide class 100 air for handling. The syringe is then connected to a patient interface or applicator, which is commonly a needle, catheter, or tubing that is then connected to a needle or catheter to the patient. For many procedures, there is some type of imaging equipment involved in guiding the applicator or effector to the correct part of the body. For example, the imaging equipment can include X-ray fluoroscopy, CT, MR, ultrasound, or an endoscope. The physician views the image and places the applicator by hand. In some neurological procedures, a stereotaxic (or stereotactic) positioner or head frame is used to guide the applicator to the target tissue, deep in the brain, based on coordinates provided by the imaging system. The patient physiological condition is often monitored for safety, especially when the patient is under general anesthesia.

Medical research has demonstrated utility of implantation of cells into the brain and central nervous system as treatment for neurodegenerative disorders such as Parkinsons, Alzheimers, stroke, or motor neuron dysfunction such as experienced, for example, by victims of spinal cord injury. As with other cell therapies, the mechanisms of repair are not well understood, but the injection of cells into damaged parenchymal tissue has been shown to recruit the body's natural repair processes and to regenerate new functional tissue as well as the cells themselves living and integrating into the tissue.

As with other cell delivery techniques described above, a long recognized, but unmet need in this field is a set of methods and devices that can provide precise, repeatable and reliable control of dosage of these therapeutic agents in actual clinical settings. Current manual approaches (as summarized above and in connection with FIG. 1) do not address all of the needs required by new procedures. For example, there are no good methods for ensuring the parameters of cell viability, including spatial distribution, cell quantity, metabolic and electrical activity, in real time during the entire implantation procedure. These variables are affected by cell storage conditions, by the fluid dynamics of an injection (for example, flow, shear stresses or forces, fluid density, viscosity, osmolarity, gas concentration), by the biocompatibility of materials, and by the characteristics of surrounding tissues and fluids.

In addition to application of cell therapies to internal tissues such a heart tissue, brain tissue and central nervous system tissue, cell therapies have also recently been applied to skin. Dermatologists have been injecting drugs into the skin for years. Recently injections of collagen, which can be thought of as a cell-less tissue, have become popular. Moreover, Intercytex of Cambridge UK has developed the ability to inject autologous dermal papilla cells for the growth of hair to treat baldness. The cells are harvested from a person, multiplied in culture, and then reimplanted into the same person. The implantation requires about 1000 injections of 1 microliter each.

Various aspects of delivery of agents such as cell to tissue and related aspects are also discussed, for example, in U.S. Pat. Nos. 5,720,720, 5,797,870, 5,827,216, 5,846,225, 5,997,509, 6,224,566, 6,231,568, 6,319,230, 6,322,536, 6,387,369, 6,416,510, 6,464,662, 6,549,803, 6,572,579, 6,599,274, 6,591,129, 6,595,979, 6,602,241, 6,605,061, 6,613,026, 6,749,833, 6,758,828, 6,796,957, 6,835,193, 6,855,132, 2002/0010428, 2002/0082546, 2002/0095124, 2003/0028172, 2003/0109849, 2003/0109899, 2003/0225370, 2004/0191225, 2004/0210188, 2004/0213756, and 2005/0124975, as well as in, PCT Published International Patent Application WO2000/067647, EP1444003, the disclosures of which are incorporated herein by reference.

Although various devices, systems and methods have been developed for the delivery of agents, including therapeutic agents, to various types of tissue, it remains desirable to develop improved devices, systems and methods for delivering agents to tissue and, particularly, for delivering therapeutic cells to tissue.

### SUMMARY

In one aspect, the present disclosure provides a fluid delivery system including a source of an injection fluid including a source outlet. The system also includes a control system including a control system inlet in fluid connection with the source outlet and a control system outlet. The control system is adapted to deliver a predetermined amount of fluid via the control system outlet upon modification of the control system outlet.

In several embodiments, the injection fluid in the source is pressurized. The source can, for example, include a plunger slidably disposed therein and a force application mechanism to place force upon the plunger and pressurize the fluid within the source.

The control system can further include a metering volume in fluid connection with a valve system. The metering volume can include a plunger slidably disposed therein.

In several embodiments, the valve system includes a first valve including a first port in fluid connection with a first port of the metering volume, a second port in fluid connection with the source outlet and a third port in fluid connection with the outlet of the control system. A second valve of the valve system includes a first port in fluid connection with a second port of the metering volume, a second port in fluid connection with the source outlet and a third port in fluid connection with the outlet of the control system. The valve system has a first state in which the first valve provides for fluid connection between the source outlet and the first port of the metering volume and the second valve provides for fluid connection between the second port of the metering volume and the control system outlet. The valve system also has a second state in which the first valve provides for fluid connection between the first port of the metering volume and the control system outlet and the second valve provides for fluid connection between the source outlet and the and the second port of the metering volume.

In several other embodiment, the metering volume can, for example, include a first port in fluid connection with the source outlet and a second port in fluid connection with a first port of the valve system. In such embodiments, a second port of the valve system can be in fluid connection with the control system outlet, and a third port of the valve system can be in fluid connection with a conduit at a first end of the conduit. A second end of the conduit is in fluid connection with the source outlet. The valve system has a first state in which the valve system provides for fluid connection between the conduit and the second port of the metering volume and a second state in which the valve system provides for fluid connection between the second port of the metering volume and the control system outlet.

In several embodiments, the plunger of the metering volume includes a forward plunger element and a rearward plunger element in connection with the forward plunger element. The forward plunger element has a surface area greater than a surface area of the rearward plunger element. The conduit can, for example, pass through the plunger.

In several other embodiments, the fluid delivery system includes a biasing element in operative connection with the plunger within the metering volume. The biasing element applies a rearward force to the plunger within the metering volume.

In still other embodiments, an actuator is attached to the control system and includes a plunger extension in operative connection with a plunger slidably disposed within a volume of the control system and a biasing element in operative connection with the plunger extension and operative to return the plunger extension to a nonactuated position. In such embodiments, the source can, for example, have a plunger slidably disposed therein. The fluid within the source need not be under pressure.

In further embodiments, the control system includes a valve system and a control mechanism in operative connection with the valve system. The valve system has a first state in which the valve system provides for fluid connection between the source outlet and the control system outlet and a second state in which the valve system prevents fluid connection between the source outlet and the control system outlet. The control mechanism is operable to control the amount of time the valve system is in the first state.

In another aspect, the present disclosure provides a cell delivery system including a source adapted to contain cells, wherein the source includes a source outlet. As described above, the cell delivery system further includes a control system including a control system inlet in fluid connection with the source outlet, a control system outlet and, alternatively, an actuator. The control system is adapted to deliver a predetermined amount of fluid via the control system outlet upon modification of the control system or activation of the actuator.

In another aspect, the present disclosure provides a fluid delivery system including a pressurized source of injection fluid, wherein the source includes a source outlet. The fluid delivery system further includes a control system including a control system inlet in fluid connection with the source outlet, a volume having a plunger slidably disposed therein, a control system outlet and, alternatively, an actuator. The volume of the control system includes a first port in fluid connection with the source outlet and a second port in fluid connection with a first port of the valve system. A second port of the valve system is in fluid connection with the control system outlet. A third port of the valve system is in fluid connection with a conduit at a first end of the conduit. A second end of the conduit is in fluid connection with the source outlet.

In a further aspect, the present disclosure provides a method of delivering a fluid to tissue including the step of injecting the fluid from a fluid delivery system including a source of an injection fluid, wherein the source includes a source outlet. The fluid delivery system also includes a control system including a control system inlet in fluid connection with the source outlet, a control system outlet and, alternatively, an actuator. The control system is adapted to deliver a predetermined amount of fluid via the control system outlet upon modification of the control system or activation of the actuator. The fluid can, for example, include cells (for example, for use in cell therapy) or contrast agent (for example, for use in diagnostic imaging).

The present disclosure, along with the attributes and attendant advantages thereof, will best be appreciated and understood in view of the following detailed description taken in conjunction with the accompanying drawings.

### BRIEF DESCRIPTION OF THE DRAWINGS

FIG. 1 illustrates a diagram of an embodiment of a currently available system and method for injection of cells.

FIG. 2A illustrates an embodiment of a fluid delivery system of the present disclosure.

FIG. 2B illustrates the fluid delivery system of FIG. 2A wherein the valve system is in a first state to effect injection and concurrent priming of a metering volume.

FIG. 2C illustrates the fluid delivery system of FIG. 2A wherein the valve system is in a second state to effect injection and concurrent refilling of a metering volume.

FIG. 3A illustrates another embodiment of a fluid delivery system of the present disclosure.

FIG. 3B illustrates the fluid delivery system of FIG. 3A wherein the valve system is in a first state to effect refilling of a metering volume.

FIG. 3C illustrates the fluid delivery system of FIG. 3A wherein the valve system is in a second state to effect injection of a metering volume.

FIG. 3D illustrates another embodiment of a fluid delivery system of the present disclosure which operates in a manner similar to that of FIG. 3A wherein the valve system is in a first state to effect refilling of a metering volume.

FIG. 3E illustrates the fluid delivery system of FIG. 3D wherein the valve system is in a second state to effect injection of a metering volume.

FIGS. 3F and 3G depict an alternative embodiment of the fluid delivery system of FIGS. 3A through 3E.

FIG. 4 illustrates another embodiment of a fluid delivery system of the present disclosure similar to the embodiment of FIG. 3A, wherein a biasing or force applying element is provided to effect refilling of the metering volume.

FIG. 5A illustrates a first side view of another embodiment of a fluid delivery system of the present disclosure.

FIG. 5B illustrates a second side view of the fluid delivery system of FIG. 5A.

FIG. 5C illustrates a front view of the fluid delivery system of FIG. 5A.

FIG. 5D illustrates a side view of a portion of the control system of the fluid delivery system of FIG. 5A wherein a plunger extension is being depressed to effect injection of fluid from the control system.

FIG. 5E illustrates another side view of a portion of the control system of the fluid delivery system of FIG. 5A wherein the plunger extension is being returned to effect refilling of fluid into the control system.

FIG. 6 illustrates another embodiment of a fluid delivery system of the present disclosure wherein a pressurized gas places force upon a plunger element.

FIG. 7 illustrates another embodiment of a fluid delivery system of the present disclosure wherein a pressurized gas places force upon a plunger element.

FIG. 8 illustrates another embodiment of a fluid delivery system of the present disclosure wherein a wearable pressurized container is in remote fluid connection with a metering volume to be injected.

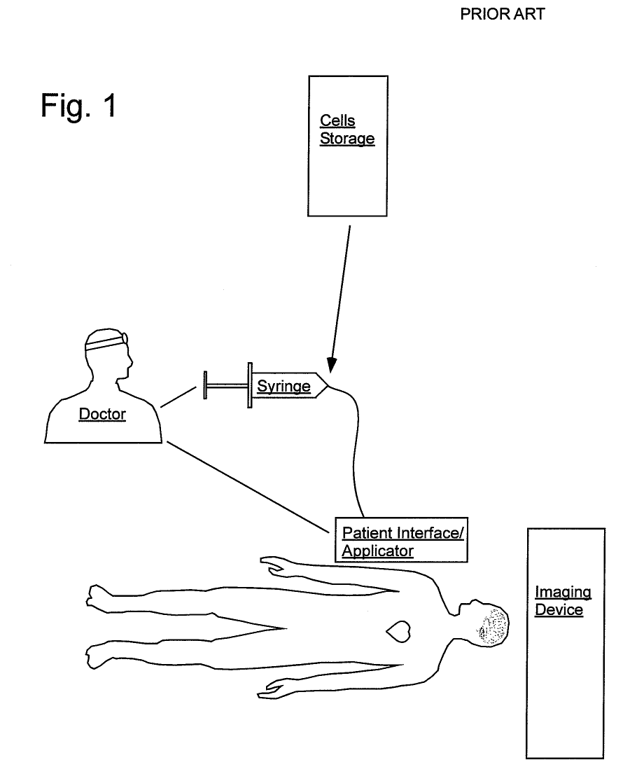

### DETAILED DESCRIPTION

In general, cell therapies are believed to work by replacing diseased or dysfunctional cells with healthy, functioning ones. However, the mechanisms of the therapies are not well understood. As described above, therapeutic treatment can involve harvesting cells from the body (such as adult stem cells) and later implanting such cells. As discussed above, the techniques are being applied to a wide range of human diseases, including many types of cancer, neurological diseases such as Parkinson's and Lou Gehrig's disease, spinal cord injuries, and heart disease. Many factors are considered when selecting an autologous or an allogeneic stem cell transplant. In general, autologous stem cell transplants (since the donor and the recipient are the same person and no immunological differences exist) are safer and simpler than allogeneic (donor cells from a healthy donor other than the recipient) stem cell transplant. However, allogenic cells can be better characterized and controlled.

In many cell therapies, a relatively small amounts of a fluid carrying cells (for example, stem cells) are repetitively injected at different injection site in the area of the therapy (for example, in external cell therapy for the heart, volumes of about 0.5 to 1.0 ml are repetitively injected at different injection sites of a beating heart). As described above, it is very difficult to achieve manual control of timing, flow rate and/or injection volume in such injections.

In several embodiments, the present disclosure provides fluid delivery systems for repetitive delivery of a predetermined amount of fluid (for example, including or carrying therapeutic cells or other agents) to one or more injection sites. The systems of the present disclosure are readily manufactured to be hand-held and/or physician worn during a procedure. Although the fluid delivery systems of the present disclosure are well suited for use in the injection of fluid incorporating one or more pharmaceutical agents, medical agents and/or biological agents, one skilled in the art appreciates that the fluid delivery systems of the present disclosure can be used in connection with many types of fluids in various fields in which fluid delivery or fluid transport is required, such as, including but not limited to, cell delivery.

For example, In the area of diagnostic imaging there is a need to deliver a metered amount of imaging agent. In nuclear imaging, a controlled amount of a radioactive isotope (FDG) is injected to the patient and a PET/CT scan is preformed. As the isotope decays (half life of FDG is 110 minutes) the volume required to deliver the same radiation activity level will have to increase correspondingly. By measuring the radiation activity level of one “slug” (metered amount) and calculating the radiation/slug the operator or a device could calculate the number of slugs required to deliver the desired radiation before a PET/CT scan is performed.

In the area of contrast delivery, a pressurized syringe could be filled with a bulk source of contrast with a metering device attached to the output. Delivering a desired volume could require a simple activation device to dispense the correct number of metered slugs to deliver the corresponding volume. For contrast dilution; a metering device could be attached to a contrast source and a metering device could be attached to a diluting source. By varying the ratio of contrast slugs to diluting slugs the concentration of the delivered contrast could be adjusted.

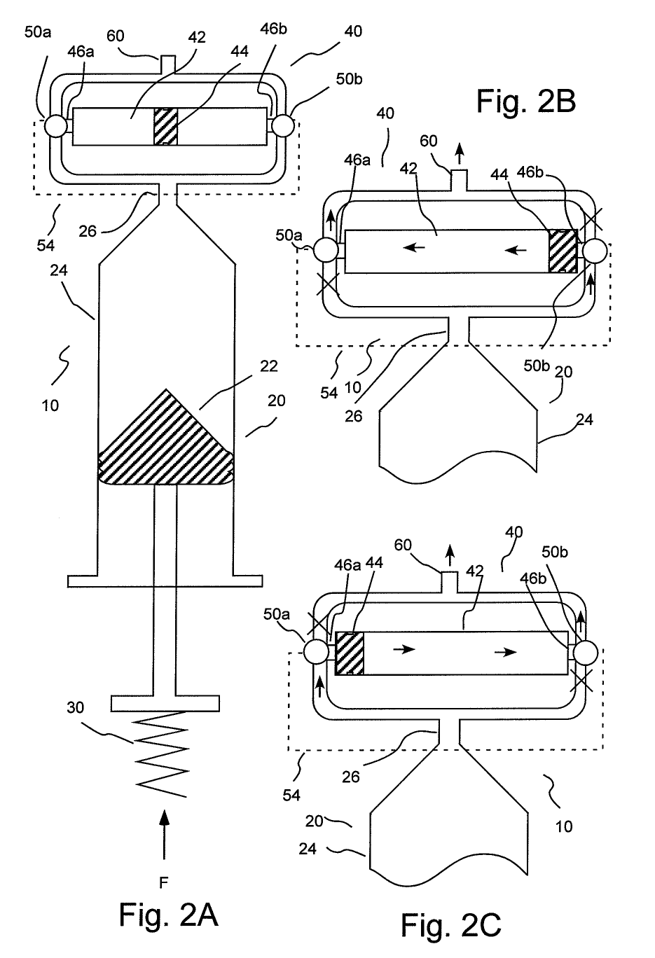

FIGS. 2A through 2C illustrates one embodiment of a fluid delivery system 10 of the present disclosure for repetitive delivery of a predetermined amount of fluid to one or more injection sites. System 10 includes a fluid reservoir such as in this embodiment, syringe 20, but any suitable fluid reservoir as known in the art could be used. Reservoir or syringe 20 includes a pressurizing mechanism in the form of a plunger 22 which is slidably disposed within a barrel 24 of syringe 20. A force F (which can, for example, have a generally constant amplitude) is applied by a force generating system 30 such as a spring system in operative connection with plunger 22. Many types of force generating systems 30 can be used in the present disclosure. Force generating system 30 can, for example, be powered by a vacuum drive, a chemical reaction, electrical power, expansion of a compressed gas, spring force or gravity. Such powering mechanisms are discussed, for example, in U.S. patent application Ser. No. 10/921,083, incorporated herein by reference, and also assigned to the assignee of the present disclosure. The application of a constant force results in a near constant flow rate of injected fluid.

An inlet of control system 40 is in fluid connection with an outlet 26 of syringe 20. In the illustrated embodiment, control system 40 includes metering volume 42 in which a slidable pressurizing mechanism, plug or plunger 44 (including, for example, a slidable elastomeric sealing member) is slidably disposed. A valve system controls flow of fluid through metering volume 42 of control system 40. In that regard, a first valve or valve system 50a is in fluid connection with a first port 46a of metering volume 42 and a second valve or valve system 50b is in fluid connection with a second port 46b of metering volume 42. In the illustrated embodiment, each of first valve system 50a and second valve system 50b is, for example, a three-port valve system such a three-way stop cock. Ports of valve system 50a are in fluid connection with syringe outlet 26, first port 46a and a system outlet 60. Ports of valve system 50b are in connection with syringe outlet 26, second port 46b and system outlet 60.

In FIG. 2B, once all air has been removed from system 10, valve system 50b is placed in a state such that (i) fluid connection is established between syringe outlet 26 and second port 46b, but fluid connection between second port 46b and system outlet 60 is blocked. Further, valve system 50a is placed in a state such that fluid connection is established between system outlet 60 and first port 46a, but fluid connection between first port 46a and syringe outlet 26 is blocked. In this state of valve system of control system 40 (that is, valve system 50a and valve system 50b), fluid is forced from syringe barrel 24 into metering volume 42 through valve system 50b and second port 46b, moving plunger 44 toward first port 46a. Fluid within metering volume 42 to the left of plunger 44 is forced from metering volume 42 (through first port 46a and valve system 50a) and exits system outlet 60. The volume injected is thus determined by the volume of metering volume 42 and the length of travel of plunger 44 therethrough (which can be adjustable). Once plunger 44 comes to rest at the left side of metering volume 42, adjacent first port 46a, fluid flow out of system outlet 60 is stopped, and the system is ready for the next injection of the same volume of fluid.

In that regard, valve system 50b is next placed in a state as illustrated in FIG. 2C such that fluid connection is blocked between syringe outlet 26 and second port 46b, but fluid connection is established between second port 46b and system outlet 60. Valve system 50a is placed in a state such that fluid connection is blocked between system outlet 60 and first port 46a, but fluid connection is established between first port 46a and syringe outlet 26. In this state of the valve system of control system 40, fluid is forced from syringe barrel 24 into metering volume 42 through valve system 50a and first port 46a, moving plunger 44 toward second port 46b. Fluid within metering volume 42 to the right of plunger 44 is forced from metering volume 42 (through second port 46b and valve system 50b) and exits system outlet 60. In the case of full travel of plunger 44 through the entire length of metering volume 42, the volume of fluid injected is generally equal to the fluid volume of metering volume 42 minus the effective volume taken up by plunger 44. Once plunger 44 comes to rest at the right side of metering volume 42, adjacent second port 46b, fluid flow out of system outlet 60 is stopped, and the system is ready for the next injection. Given the continuous application of force from force generating system 30, and the repeated manipulation of valve systems 50a and 50b, the above-described process can be repeated until the total volume of fluid within syringe barrel 24 is exhausted with each separate injection delivering the same amount of volume as set by metering volume 42. Generally simultaneous control of valve systems 50a and 50b can, for example, be achieved via an actuator 54, which can, for example, include mechanical and/or electromechanical control mechanisms as known in the art. These mechanisms may be located in close proximity to valve systems 50a and 50b or may allow for remote operation (such as, for example, a foot switch). Well known adjustable stop mechanisms, for example a thumb screw, threaded insert or other adjustable device as known in the art, can be provided within metering volume 42 to limit the travel of plunger 44 and thereby control the volume of fluid injected into a patient.

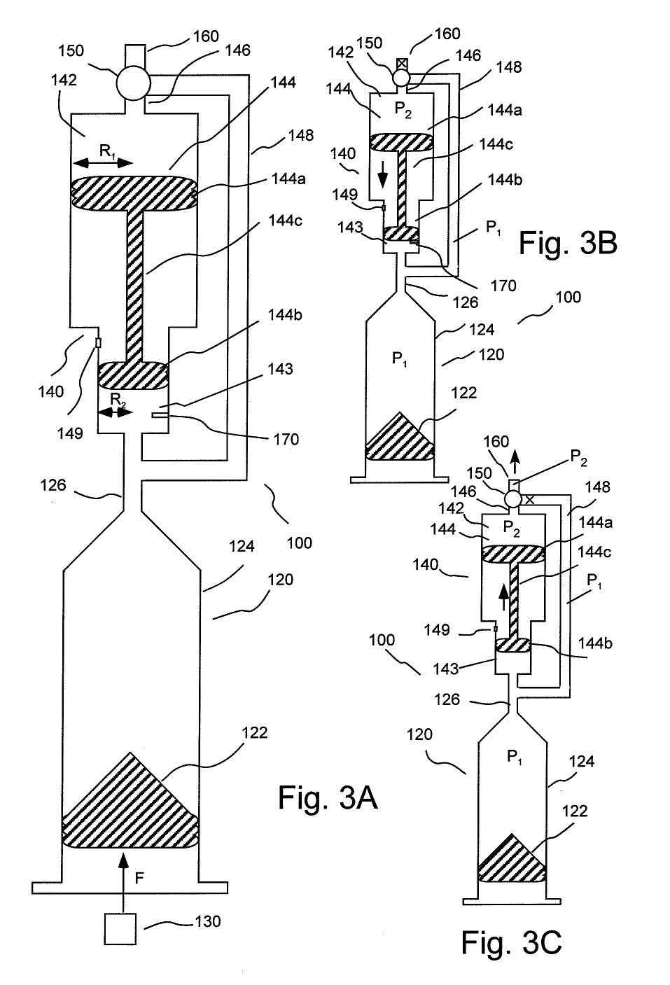

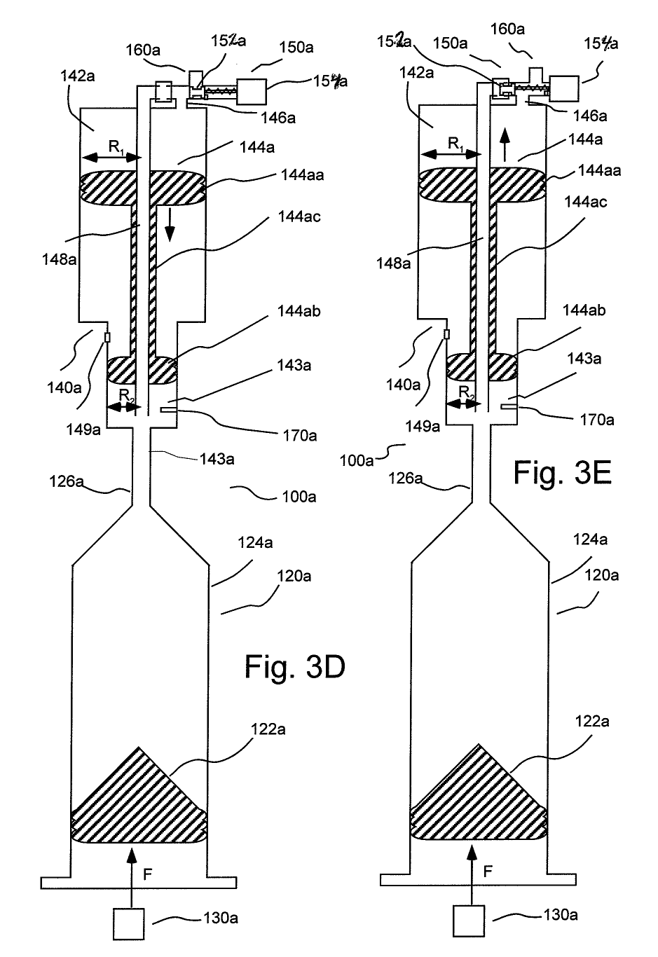

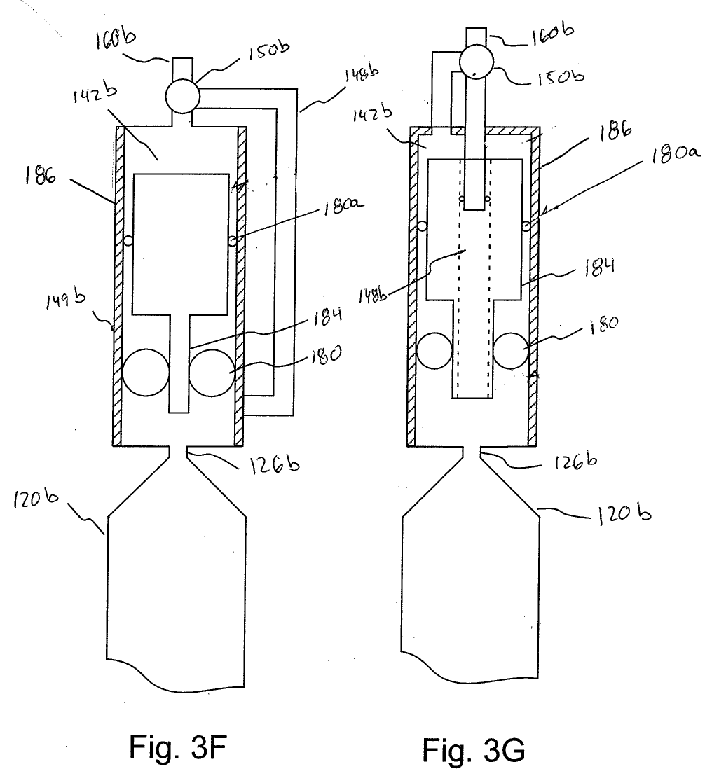

FIGS. 3A through 3C illustrate another embodiment a fluid delivery system 100 of the present disclosure for repetitive delivery of a predetermined amount of fluid to one or more injection sites. System 100 includes a fluid reservoir such as in this embodiment, syringe 120 but any suitable fluid reservoir as known in the art could be used. Reservoir or syringe 120 includes a pressurizing mechanism in the form of a plunger 122 which is slidably disposed within a barrel 124 of syringe 120. As with prior embodiments previously discussed, a force F (which can, for example, have a constant amplitude) is applied by a force generating system 130 in operative connection with plunger 122.

A control system 140 is in fluid connection with an outlet 126 of syringe 120. In the illustrated embodiment, control system 140 includes a metering volume 142 in which a plunger assembly 144 is slidably disposed. Metering volume 142 can, for example, be generally cylindrical in shape. Plunger assembly 144 includes a first sealing plunger element 144a (for example, including an elastomeric material) slidably disposed within a metering volume 142. Plunger assembly 144 also includes a second sealing plunger element 144b (for example, including an elastomeric material) slidably disposed within a second volume 143 that is in fluid connection with metering volume 142. First sealing plunger element 144a has a radius R₁ that is larger than a radius R₂ (see, for example, FIG. 3A) of second sealing plunger element 144b. First sealing plunger element 144a is connected to second sealing plunger element 144b by an extending member 144c. A valve system (for example, a three-port valve system such as a three-way stop cock) 150 includes a first port that is in fluid connection with a port 146 of metering volume 142. Valve system 150 includes a second port that is in fluid connection with system outlet 160 and a third port that is in fluid connection with a first end of conduit 148. A second end of conduit 148 is in fluid connection with syringe outlet 126. A vent 149 can be provided in fluid connection with metering volume 142 and volume 143 because of pressure differences created. Because the surface area of first sealing plunger element 144a is larger than the surface area of second sealing plunger element 144b, pressure may build up within the system upon retraction of plunger assembly 144 unless a vent such as vent 149 is provided.

Instead of a three-way stop cock as valve system 150, other embodiments can include a modified TRAC™ valve available from Qosina of Edgewood, N.Y. under product number QOS5402597N and manufactured by B. Braun (see, for example, U.S. Pat. No. 5,064,168 and U.S. Pat. No. 5,228,646, the disclosure of which are incorporated herein by reference). The valve as available from Qosina is a two-port, linear TRAC valve. In the present disclosure, the third port of valve system 150 (which is in connection with the first end of conduit 148) was formed by drilling a hole into the valve as available from Qosina.

As illustrated in FIG. 3B, once all air has been removed from system 100, when valve system 150 is placed in a state such that it is closed to outlet 160 (and thereby closed to atmospheric pressure), while providing for fluid connection between conduit 148 and port 146 of metering volume 142, the pressures at each port are equal, i.e., P₁=P₂. In this state, because radius R₁ is larger than R₂, the force on first sealing plunger element 144a (because of its larger surface area) is larger than the force on second sealing plunger element 144b. In that regard, the force on each of plunger element 144a and plunger element 144b is equal to pressure multiplied by the surface area of the plunger element as follows:

Force on first sealing plunger element 144a=P₂×π(R₁)²

Force on second sealing plunger element 144b=P₁×π(R₂)²

Because P₁=P₂ and R₁ is greater than R₂, the force on first plunger element 144a is greater than the force on second plunger element 144b. The greater force on first plunger element 144a results in rearward movement of plunger assembly 144 (that is, movement toward syringe 120). Rearward movement of plunger assembly 144 results in filling of metering volume 142 with fluid from syringe/reservoir 120 via conduit 148. At least one adjustable stop 170 can be provided, for example, within volume 143 to limit the movement of plunger assembly 144 to control the volume of fluid drawn into metering volume 142.

As illustrated in FIG. 3C, valve system 150 is then placed in a state such that metering volume 142 is placed in fluid connection with system outlet 160 and conduit 148 is blocked from fluid connection with system outlet 160 and metering volume 142. In this state, pressure P₂ is equal to atmospheric pressure. Pressure P₁ (the pressure of the pressurized fluid within syringe 120) is greater than pressure P₂ such that forward force on second plunger element 144b is greater than the rearward force on first plunger element 144a (that is, P₁×π(R₂)₂ is greater than P₂×π(R₁)²). This pressure differential results in forward movement of plunger assembly 144 and delivery/injection of metering volume 142 of fluid forward of first plunger element 144a to the patient.

Once the fluid is injected, valve system 150 can once again be placed in the state illustrated in FIG. 3B, resulting in automatic refilling of metering volume 142 with fluid. The process can be repeated to repeatedly inject a controlled volume of fluid. Given the continuous application of force from force generating system 130, and the repeated manipulation of valve system 150, the above-described process can be repeated until the total volume of fluid within syringe barrel 124 is exhausted with each separate injection delivering the same amount of volume as set by metering volume 142.

FIGS. 3D and 3E illustrate another embodiment of a fluid delivery system 100a of the present disclosure for repetitive delivery of a predetermined amount of fluid to one or more injection sites. In many respects, fluid delivery system 100a operates in the same or a similar manner to fluid delivery system 100 as shown in FIGS. 3A through 3C and like components are numbered in a corresponding manner with the designation “a” added to the component designations of FIGS. 3D and 3E.

In the embodiment of FIGS. 3D and 3E, conduit 148a is positioned within volume 140a and passes through plunger assembly 144a. Similar to the operation of system 100, given a constant force generated by force generating system 130a, when valve system 150a is placed in a first state illustrated in FIG. 3D such that it is closed to outlet 160a (and thereby closed to atmospheric pressure), while providing for fluid connection between conduit 148a and port 146a of metering volume 142a, P₁=P₂. In this state, because radius R₁ is larger than R₂, the force on first plunger element 144aa (because of its larger surface area) is larger than the force on second plunger element 144ab.

The greater force on first plunger element 144aa results in rearward movement of plunger assembly 144a (that is, movement toward syringe 120a). Rearward movement of plunger assembly 144a results in filling of metering volume 142a with fluid from syringe/reservoir 120a via conduit 148a.

As illustrated in FIG. 3E, valve system 150a is then placed in a second state such that metering volume 142a is placed in fluid connection with system outlet 160a and conduit 148a is blocked from fluid connection with system outlet 160a and metering volume 142a. In this state, pressure P₂ is equal to atmospheric pressure. Pressure P₁ (the pressure of the pressurized fluid within syringe 120a) is greater than pressure P₂. This pressure differential and the resulting difference in forces on first plunger element 144aa and second plunger element 144ab results in forward movement of plunger assembly 144a and delivery/injection of metering volume 142a of fluid forward of first plunger element 144aa to the patient.

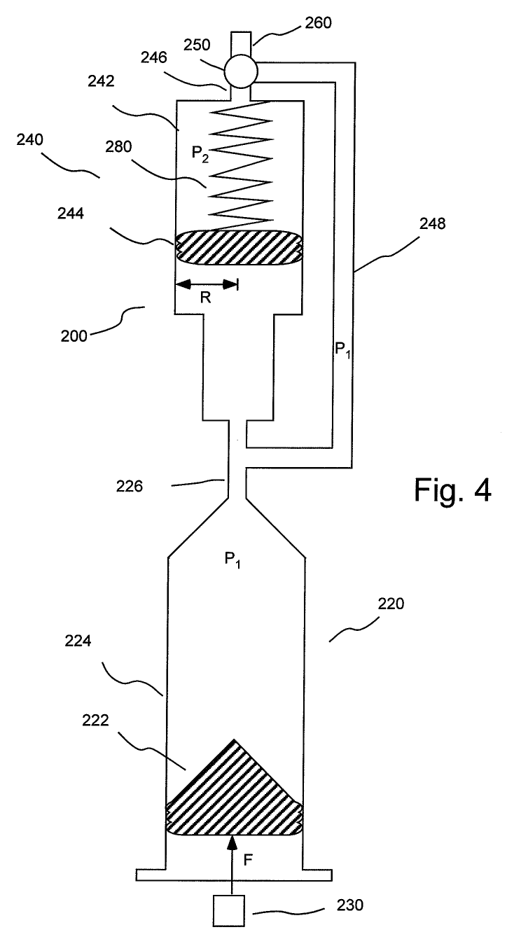

As with other embodiments, valve system 150a was formed by modifying TRAC™ valve available from Qosina of Edgewood, N.Y. under product number QOS5402597N and manufactured by B. Braun (see, for example, U.S. Pat. No. 5,064,168 and U.S. Pat. No. 5,228,646, the disclosure of which are incorporated herein by reference). The valve as available from Qosina is a two-port, linear TRAC valve. In the present disclosure, the third port of valve system 150a (which is in connection with the first end of conduit 148a) was formed by drilling a hole into the valve as available from Qosina. As illustrated in FIGS. 3D and 3E, valve system 150a includes a sealing plug member 152a in operative connection with an actuator 154a. Controlling the position of actuator 154a, and thereby plug member 152a, controls whether valve system 150a is in the first state or the second state as illustrated in FIGS. 3D and 3E. Valve system 150a can be used in connection with other fluid delivery systems of the present disclosure as, for example, illustrated in FIGS. 3A through 3C and in FIG. 4. In the first state, plug member 152a blocks the second port of valve system 150a (and, thereby, control system outlet 160a). In the second state, plug member 152a blocks the third port of the valve system 150a (and, thereby, the first end of conduit 148a) and this functionality is easily transferable to other embodiments of the present disclosure.

FIGS. 3F and 3G represent an alternative embodiment of the present disclosure where sealing members 180 and 180a operate to control the pressure within volume 140b. Rather than the typical rubber cover that is part of the plungers in other embodiments, two sets of sealing members of varying sizes 180 and 180a allow for the movement of slidable member or piston 184. Sealing members 180 can be fixed to the inside of container 186. Sealing members 180a can also be fixed to the inside of container 186 or, alternatively, to slidable member 184. As with other embodiments disclosed herein, valve system 150b can be manipulated to allow for filling of metering volume 142b from syringe 120b via syringe outlet 126b which is in fluid connection with conduit 148b. Through further manipulation of valve system 150b, the fluid is then delivered to the patient through system outlet 160b. Vent 149b also allows for the proper pressurization of the volume between sealing members 180

FIG. 4 illustrates another embodiment a fluid delivery system 200 of the present disclosure for repetitive delivery of a predetermined amount of fluid to one or more injection sites. System 200, which operates in a number of respects in a similar manner to the earlier described system 100, includes a fluid reservoir of pressurized fluid in the form of a syringe 220. Reservoir or syringe 220 includes a pressurizing mechanism in the form of a plunger 222 which is slidably disposed within a barrel 224 of syringe 220. A force F (which can, for example, have a constant amplitude) is applied by a force generating system 230 in operative connection with plunger 222.

A control system 240 is in fluid connection with an outlet 226 of syringe 220. In the illustrated embodiment, control system 240 includes a metering volume 242 in which a plunger 244, forms a slidable, sealing engagement with the internal walls of volume 242, and is slidably disposed. Volume 242 can, for example, be generally cylindrical in shape. Valve system 250 (for example, a three-port valve system such as a three-way stop cock or valve system 150a of FIGS. 3D and 3E) includes a first port in fluid connection with a port 246 of metering volume 242. Valve system 250 includes a second port that is in fluid connection with system outlet 260 and a third port in fluid connection with a first end of conduit 248. A second end of conduit 248 is in fluid connection with syringe outlet 226.

When valve system 250 is placed in a first state such that it is closed to outlet 260 (and thereby closed to atmospheric pressure), while providing for fluid connection between conduit 248 and port 246 of metering volume 242, the pressures at each port are equal, i.e., P₁=P₂. The forward force on plunger 244 is P₁×πR². The rearward force on plunger 244 is P₂×πR² plus the force exerted by a biasing element such as spring 280. In the state wherein P₁=P₂, the rearward force on plunger 244 exceeds the forward force on plunger 244 by an amount equal to the rearward force exerted by spring 280, and plunger 244 is forced rearward (toward syringe 120) causing metering volume 242 to be filled with the pressurized fluid via conduit 248 (in an amount dependent upon the linear distance of rearward travel of plunger 244, which can be adjustable).

Valve system 250 can then be placed in a second state such that metering volume 242 is place in fluid connection with system outlet 260 and conduit 248 is blocked from fluid connection with system outlet 260 and metering volume 242. In this state, pressure P₂ is equal to atmospheric pressure. Pressure P₁ (the pressure of the pressurized fluid within syringe 220) is greater than pressure P₂. The forward force on plunger 244 is now greater than the rearward force on plunger 244. This force differential results in forward movement of plunger 244 (overcoming the force applied to plunger 244 by spring 280) and delivery/injection of metering volume 242 of fluid forward of plunger 244 to the patient.

Once the fluid is injected, valve system 250 can once again be placed in the first state, resulting in automatic refilling of metering volume 242 with pressurized fluid. The process can be repeated to repeatedly inject a controlled volume of fluid. Given the continuous application of force from force generating system 230, and the repeated manipulation of valve system 250, the above-described process can be repeated until the total volume of fluid within syringe barrel 224 is exhausted with each separate injection delivering the same amount of volume as set by metering volume 242. As with other embodiments, an actuator (not shown) may be employed to allow for easy manipulation of valve system 250.

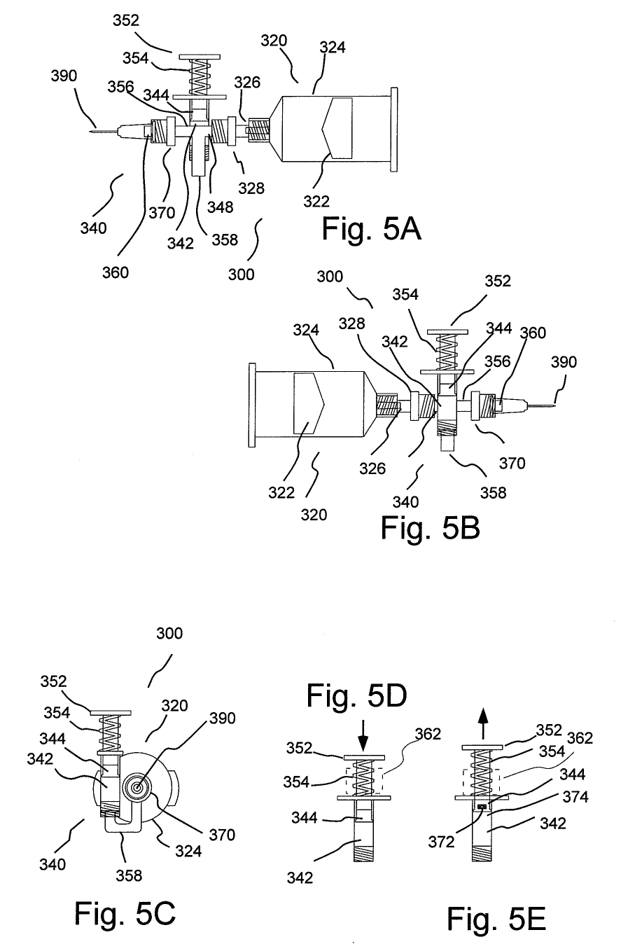

FIGS. 5A through 5E illustrate another embodiment a system 300 of the present disclosure for repetitive delivery of a predetermined amount of fluid to one or more injection sites. System 300 includes a fluid reservoir in the form of a syringe 320 but any suitable fluid reservoir as known in the art could be used. Reservoir or syringe 320 includes a plunger 322 which is slidably disposed within a barrel 324 of syringe 320.

A control system 340 includes a valve system to control fluid flow therethrough. In that regard, control system 340 is in fluid connection with an outlet 326 of syringe 320 via an intervening one-way or check valve 328 (which can, for example, be attached to syringe 320 and to control system 340 via luer connections as known in the art). Check valve 328 allows fluid to flow into inlet port 348 of control system 340, but prevents fluid from flowing rearward from control system 340 into syringe 320.

Control system 340 includes a metering volume 342 in which a sealing plunger 344 is slidably positioned. The position of plunger 344 within metering volume 342 is controlled by the position of a plunger extension 352. A biased or force applying return mechanism such as a spring 354 is in operative connection with plunger extension 352.

To inject fluid from control system 340 into a patient (for example, via a needle 390 in fluid connection with system outlet 360) plunger extension 352 is forced downward through metering volume 342. Needle 390 is in fluid connection with outlet 360 via an intervening one-way or check valve 370, which allows fluid to flow from outlet 360 to needle 390, but prevents fluid flow from needle 390 back through outlet 360 into control system 340. The pressure created by activation of plunger extension 352 causes a volume of fluid equal to the volume displaced from metering volume 342 by passage of plunger 344 therethrough to be injected into the patient via needle 390.

After force is removed from plunger extension 352, spring 354 causes plunger extension to move upward so that fluid is automatically drawn into control system 340 from syringe 320. The vacuum created within metering volume 342 by retraction of plunger 344 causes plunger 322 of syringe 320 to be drawn toward control system 340. In the embodiment of FIGS. 5A through 5E, no force need be applied to plunger 322 to achieve this result. The process can be repeated to repeatedly inject a controlled volume of fluid into a patient. The range of motion of plunger extension 352 can, for example, be controlled (for example, via use of a collar 362 of adjustable length as illustrated in FIGS. 5D and 5E) to control the volume of fluid injected.

In the illustrated embodiment, metering volume 342 is in fluid connection with a generally linear length of conduit 356 via a generally U-shaped conduit 358. A first end of conduit 356 forms control system inlet 348 and a second end of conduit 356 forms control system outlet 360. A vent hole 372 and vent hole filter 374 (see FIG. 5E) can be provided in fluid connection with, for example, metering volume 342 to enable removal of air (or priming) of system 300 prior to the repeated injection of fluid into a patient. In the embodiment depicted in FIGS. 5A through 5E, outlet 326 is in direct fluid connection with check valve 328. In other embodiments (not shown), outlet 326 may be connected to check valve 328 via a length of tubing.

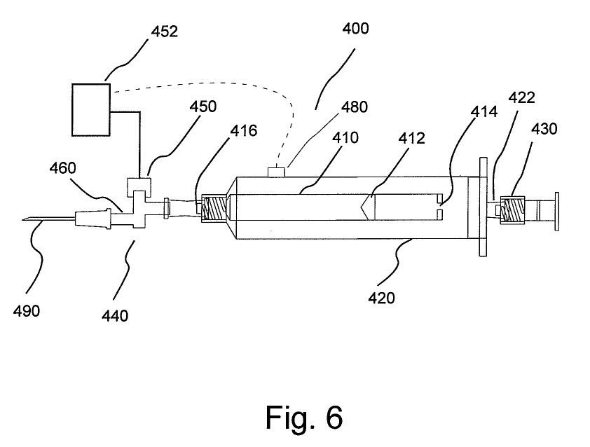

FIG. 6 illustrates another embodiment of a fluid delivery system 400 of the present disclosure. System 400 includes a volume or reservoir 410 in which an injection fluid (for example, including cells for cell therapy) is contained. A pressurizing mechanism such as a sealing, slidable plunger 412 is in operative connection with volume 410 to pressurize the fluid therein. In that regard, force is applied to slidable plunger 412 to pressurize the fluid within volume 410. As with each of the embodiments disclosed herein, force can be applied, for example, as powered by a vacuum drive, a chemical reaction, electrical power, expansion of a compressed gas, spring force or gravity as, for example, disclosed in U.S. patent application Ser. No. 10/921,083. In the illustrated embodiment, volume 410 is contained within or encompassed by a container or volume 420. Volume 420 also contains a pressurized gas such as pressurized carbon dioxide (CO₂) that is introduced into volume 420 through check valve 430, which enables the pressure within volume 420 to maintain, or be repressurized, during injection. The pressurized gas is in fluid connection with a rearward end of plunger 412 via a port 414.

A control system 440 is in fluid connection with an outlet port 416 of volume 410. Control system 440 includes a valve system 450 (for example, a TRAC™ valve product number QOS5402597N available from Qosina of Edgewood, N.Y. Activation (depression) of valve 450 results in placing outlet 460 (and attached needle 490) in fluid connection with outlet port 416 of volume 410 so that fluid is injected into a patient. Fluid will be injected via needle 490 at a relatively constant flow rate until valve system 450 is placed in a non-actuated state (released). Control system 440 can include an actuating mechanism 452 that is operable to actuate valve system 450 for a predetermined time so that a predetermined volume of fluid can be injected upon each activation. Feedback of pressure within volume 420 can be provided from a pressure sensor 480 to assist in ensuring that flow rate and/or volume injected is maintained relatively constant with changing pressure within volume 420. Alternatively, one or more algorithms can be used as known in the art (for example, based upon the number of injections made) to calculate the change in pressure within volume 420.

To introduce or refill volume 410, check valve 430 can, also remove a vacuum when applied to port 422 to draw plunger 412 rearward within volume 410 and thus draw fluid into volume 410 via outlet 416. Alternatively, needle 490 or control system 440 can be removed so that fluid can be forced into volume 410 via outlet 416. When check valve 430 is in fluid connection with inlet port 422, volume 420 can be charged with pressurized gas therethrough.

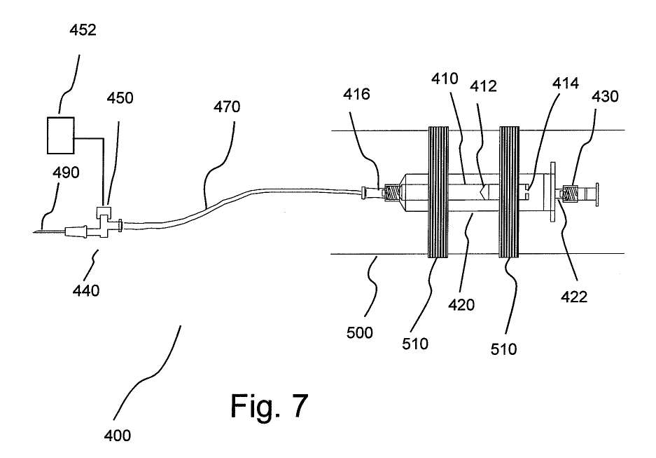

In the embodiment of FIG. 6, outlet 416 is generally in direct connection with control system 440. In FIG. 7, outlet 416 is in fluid connection with control system 440 via a length of flexible tubing 470. This enables, for example, the attachment of volume 410 to an arm 500 of a physician. This attachment can, for example, be effected using straps 510, which can, for example, include hook-and-loop type fastening systems as know in the art. Thus the physician can hold the relatively small control system 440 during an injection procedure while having general freedom of movement. The embodiment of FIG. 7 may, for example, provide increased flexibility in attaining access to certain injection sites as compared to the embodiment of FIG. 6 and can be easily adapted into other embodiments of the present disclosure. For example a metering volume (not shown) could be located in close proximity to control system 440 (such as in FIGS. 5A through 5E) with fluid refilling same after each injection via tubing 470.

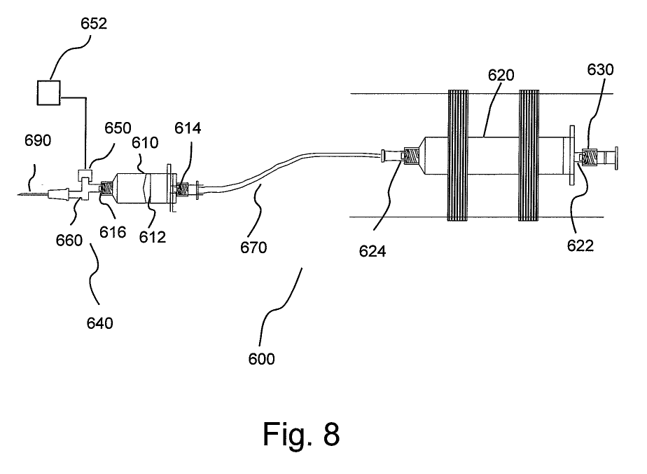

FIG. 8 illustrates another embodiment of a fluid delivery system 600 of the present disclosure. In many respects, system 600 operates in a manner similar to system 400 illustrated in FIG. 7. However, in the embodiment of FIG. 8, a pressurizing container of volume 620 is positioned in remote fluid connection with a volume 610 (which includes an injection fluid therein).

As described above in connection with pressurizing volume 420, pressurizing volume 620 includes a one-way or check valve 630 in connection with an inlet 622 through which volume 620 can be charged with a pressurized gas (for example, CO₂). An outlet 624 of pressurizing volume 620 is in fluid connection with an inlet 614 of volume 610 such that the pressurized gas results in a forward acting force on a rearward side of a plunger 612 slidably positioned within volume 610. An outlet 616 of volume 610 is in fluid connection with control system 640, which operates in the same manner as control system 440, or other control systems disclosed herein. In that regard, control system 640 includes valve system 650 as disclosed in other embodiments (for example, a TRAC™ valve). Activation (depression) of valve 650 results in placing outlet 660 (and attached needle 690) in fluid connection with outlet port 616 of volume 610 so that fluid is injected into a patient. Fluid will be injected via needle 690 at a relatively constant pressure until valve system 650 is placed in a non-actuated state (released). As described above, control system 640 can include an actuating mechanism 652 that is operable to actuate valve system 650 for a predetermined time so that a predetermined volume of fluid can be injected upon each activation.

As volume 610 is in close proximity to control system 640 in the embodiment of FIG. 8, waste of injection fluid that can be associated with intervening tubing can be reduced or eliminated.

The foregoing description and accompanying drawings set forth the preferred embodiments of the disclosure at the present time. Various modifications, additions and alternative designs will, of course, become apparent to those skilled in the art in light of the foregoing teachings without departing from the scope of the disclosure. The scope of the disclosure is indicated by the following claims rather than by the foregoing description. All changes and variations that fall within the meaning and range of equivalency of the claims are to be embraced within their scope.

What is claimed is:

1. A fluid delivery system comprising a source of an injection fluid, the source including a source outlet, and a control system comprising a control system inlet in fluid connection with the source outlet, a control system outlet, the control system being adapted to deliver a predetermined amount of fluid via the control system outlet upon modification of the control system outlet.

2. The fluid delivery system of claim 1 wherein the injection fluid in the source is pressurized.

3. The fluid delivery system of claim 2 wherein the source comprises a plunger slidably disposed therein and a force application mechanism to place force upon the plunger.

4. The fluid delivery system of claim 2 wherein the control system further comprises a metering volume in fluid connection with a valve system in fluid connection with the control system inlet and the control system outlet.

5. The fluid delivery system of claim 4 wherein the metering volume comprises a plunger slidably disposed therein.

6. The fluid delivery system of claim 5 wherein the valve system comprises a first valve, the first valve comprising a first port in fluid connection with a first port of the metering volume, a second port in fluid connection with the source outlet and a third port in fluid connection with the outlet of the control system, and a second valve comprising a first port in fluid connection with a second port of the metering volume, a second port in fluid connection with the source outlet and a third port in fluid connection with the outlet of the control system, the valve system having a first state in which the first valve provides for fluid connection between the source outlet and the first port of the metering volume and the second valve provides for fluid connection between the second port of the metering volume and the control system outlet and a second state in which the first valve provides for fluid connection between the first port of the metering volume and the control system outlet and the second valve provides for fluid connection between the source outlet and the and the second port of the metering volume.

7. The fluid delivery system of claim 5 wherein the metering volume comprises a first port in fluid connection with the source outlet and a second port in fluid connection with a first port of the valve system, a second port of the valve system being in fluid connection with the control system outlet, a third port of the valve system being in fluid connection with a conduit at a first end of the conduit, and a second end of the conduit being in fluid connection with the source outlet.

8. The fluid delivery system of claim 7 wherein the valve system has a first state in which the valve system provides for fluid connection between the conduit and the second port of the metering volume and a second state in which the valve system provides for fluid connection between the second port of the metering volume and the control system outlet.

9. The fluid delivery system of claim 8 wherein the plunger of the metering volume includes a forward plunger element and a rearward plunger element in connection with the forward plunger element, the forward plunger element having a surface area greater than a surface area of the rearward plunger element.

10. The fluid delivery system of claim 9 wherein the conduit passes through the plunger.

11. The fluid delivery system of claim 8 further comprising a biasing element in operative connection with the plunger within the metering volume, the biasing element applying a rearward force to the plunger within the metering volume.

12. The fluid delivery system of claim 1 further comprising an actuator in operative connection with the control system and a plunger extension in operative connection with a plunger slidably disposed within a volume of the control system and a biasing element in operative connection with the plunger extension and operative to return the plunger extension to a nonactuated position.

13. The fluid delivery system of claim 3 wherein the control system comprises a valve system and a control mechanism in operative connection with the valve system, the valve system having a first state in which the valve system provides for fluid connection between the source outlet and the control system outlet and a second state in which the valve system prevents fluid connection between the source outlet and the control system outlet, the control mechanism is operable to control the amount of time the valve system is in the first state.

14. The fluid delivery system of claims 1 or 12 wherein the source of injection fluid is adapted to contain cells.

15. The fluid delivery system of claim 2 wherein the pressure creates a constant force on the source outlet generating a constant flow rate for the injection fluid.

16. The fluid delivery system of claim 2 wherein the source of injection fluid is in remote fluid connection with the control system.

17. The fluid delivery system of claim 4 further comprising a slidable member and a plurality of sealing members that operate to isolate the metering volume as the slidable member moves between the sealing members.

18. A method of delivering a fluid to tissue comprising injecting the fluid from a fluid delivery system comprising a pressurized source of an injection fluid, the source including a source outlet, and a control system comprising a control system inlet in fluid connection with the source outlet, a control system outlet and an actuator, the control system being adapted to deliver a predetermined amount of fluid via the control system outlet upon activation of the actuator.

19. The method of claim 18 wherein the fluid comprises cells.
